(19) United States
(12) Patent Application Publication
(10) Pub. No.: US 2017/0172421 A1
(43) Pub. Date: Jun. 22, 2017

(54) PHYSIOLOGICAL CHARACTERISTIC MEASUREMENT SYSTEM

(71) Applicants: Nadine L. Dabby, Palo Alto, CA (US); Adel A. Elsherbini, Chandler, AZ (US); Sasha Oster, Chandler, AZ (US); Braxton Lathrop, Lake Oswego, OR (US); Aleksandar Aleksov, Chandler, AZ (US); Feras Eid, Chandler, AZ (US)

(72) Inventors: Nadine L. Dabby, Palo Alto, CA (US); Adel A. Elsherbini, Chandler, AZ (US); Sasha Oster, Chandler, AZ (US); Braxton Lathrop, Lake Oswego, OR (US); Aleksandar Aleksov, Chandler, AZ (US); Feras Eid, Chandler, AZ (US)

(21) Appl. No.: 14/971,800

(22) Filed: Dec. 16, 2015

(51) Int. Cl. A61B 5/0205; G06F 3/01; A61B 5/00

(52) U.S. Cl. CPC A61B 5/0205; A61B 5/6804; G06F 3/017; A61B 5/08

(57) ABSTRACT

A sensor assembly configured to monitor one or more physiological characteristics includes a deformable substrate. The deformable substrate includes a body side interface. Substrate conductive traces are coupled with the deformable substrate. Two or more physiological sensor elements are coupled with the deformable substrate. The two or more physiological sensor elements include at least first and second sensor elements. The first sensor element includes a first piezo element in a first orientation along the deformable substrate, the first sensor element is electrically coupled with the substrate conductive traces. The second sensor element includes a second piezo element in a second orientation along the deformable substrate different than the first orientation, the second sensor element is electrically coupled with the substrate conductive traces.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the the drawings that form a part of this document: Copyright Intel Corporation, Santa Clara, Calif. All Rights Reserved.

### TECHNICAL FIELD

Embodiments described herein generally relate to the measurement and identification of physiological characteristics.

### BACKGROUND

User interface devices including remotes, smart phones, wearable devices (bracelets and watches) or the like include accelerometers configured to detect movement of the device. In some examples, accelerometers are used to reorient the view of a screen, initiate powering of the screen (e.g., when moved from rest) or the like. In other examples, accelerometers in pedometers or other wearable devices detect movement of the body and log the movement as steps.

In still other examples, a plurality of sensors of positioned on the body to measure overall movement of the user. For instance, accelerometer units are buckled to the body or incorporated into that are worn (e.g., for motion capture). Each of the accelerometers is powered with its own battery or a system battery on the user or at rest at a nearby location (e.g., on a floor, table or the like). The accelerometers include transmitters and power cables that broadcast motion to a central processor.

### BRIEF DESCRIPTION OF THE DRAWINGS

FIG. 1 is a schematic view of one example of a garment including a physiological characteristic measurement system.

FIG. 2 is a schematic view of the physiological characteristic measurement system in another example of a garment.

FIG. 3 is a top view of one example of a sensor assembly.

FIG. 4 is a cross sectional view of the sensor assembly of FIG. 3.

FIG. 5 is a schematic control diagram of the physiological characteristic measurement system.

FIG. 6 is a block diagram showing one example of a method for identifying a physiological characteristic.

FIG. 7 is a block diagram showing one example of a method for making a physiological characteristic measurement system.

### DESCRIPTION OF EMBODIMENTS

The present inventors have recognized, among other things, that a problem to be solved can include accurately identifying user gestures, magnitude of gestures and the like for interaction and control of connected systems. For instance, accelerometer systems as described herein are housed in smart phones and remotes (e.g., electronic remotes, game controllers or the like) and are used to provide gross control of devices including powering on of screens with movement, reorienting of a view on the screen or the like. Similarly, wearable devices include accelerometers or the like for detection of ambulatory steps in the manner of a pedometer for fitness analysis and record keeping. In some examples an accelerometer at a location, such as the wrist and provides limited information about movement. Further, accelerometers are bulky, cumbersome to incorporate into textiles and require a power source (through cables or individual power units associated with the accelerometers). These devices fail to provide recognition and identification of physiological characteristics and magnitude of the same, such as human gestures. For instance, complex human gestures including intricate hand, wrist and arm movements are not identified.

The present subject matter can help provide a solution to this problem, such as by sensor assemblies worn by a user and configured to measure physiological movement through deformation of at least one sensor element. In some examples, the sensor element includes a piezo element (e.g., a piezo-resistive or piezo-electric element). The sensor assembly is worn in close proximity to the body of the user, for instance, as a garment, patch, cuff, jewelry article or the like. The piezo element is configured to sense deformation including deformation at locations of the body caused by movement of limbs, digits, respiration, heart beats or the like. An associated controller interprets the deformation and provides one or more of a direction of the motion and its magnitude (a vector). In another example, a plurality of sensor assemblies are incorporated into a garment including a suit, wearable garment, cuffs or the like at varying locations on the user. The controller receives measured deformations from each of the sensor assemblies and analysis of the deformations at multiple locations facilitates the identification of gestures and body movements as well as the respective magnitude. In still another example, the sensor assemblies each optionally include a plurality of sensor elements, such as first and second piezo elements at differing orientations. Deformation measured at the location of the first and second piezo elements allows for the determination of magnitude and direction of the movement at the limb as it causes component deformation in each of the piezo elements (e.g., along x and y-axis oriented elements). Optionally, a supplement sensor, such as a piezo element an accelerometer or the like is used in combination with other piezo elements as a filter (to reduce noise) for the output of the base piezo elements.

The present inventors have recognized, among other things, that another problem to be solved can include minimizing the encumbrance and profile of a physiological characteristic system configured to identify one or more physiological characteristics. The accelerometers of accelerometer systems (e.g., provided in a suit or buckled to a user) include subassemblies having individual accelerometers, transmitters and in at least some examples dedicated power sources. Alternatively, power cables are distributed to each of the accelerometer subassemblies. These suits and their associated accelerometers are accordingly bulky, heavy and expensive.

The present subject matter can help provide a solution to this problem, such as by sensor assemblies worn by a user including conductive traces and one or more sensor elements that are compact and thereby provide a minimized profile. In an example, the sensor assemblies described herein are formed by providing conductive traces and piezo elements with inks that are cured on a deformable substrate, such as a textile or elastomer. The inks are printed on the deformable substrate by way of screen printing, sputtering, propulsion of ink (e.g., as in ink jet printing) or the like. Optionally, a shell such as a layer of elastomer is provided over top of the cured traces and piezo elements for protection from the elements, wear, washing and drying or the like. In still other examples, interconnecting conductive traces are provided in garments between sensor assemblies and a controller (e.g., positioned in a garment tag, patch or the like). The interconnecting conductive traces are optionally formed with conductive threading, cured inks in the manner of the conductive traces described herein or the like.

Sensor assemblies as described herein are compact, lightweight and have a minimized profile. Accordingly the sensor assemblies are readily incorporated into clothing, cuffs, jewelry or the like. Optionally, the sensor assemblies are integrally formed with the fabric of the clothing or applied in the manner of an adhered assembly (e.g., iron-on patch). Garments including the sensor assemblies, controller and interconnecting conductive traces thereby provide a compact profile resembling regular garments such as clothing, jewelry, cuffs or the like while still providing enhanced detection and identification of physiological characteristics such as gestures, respiration, heart contraction/relaxation.

This overview is intended to provide an overview of subject matter of the present patent application. It is not intended to provide an exclusive or exhaustive explanation of the invention. The detailed description is included to provide further information about the present patent application.

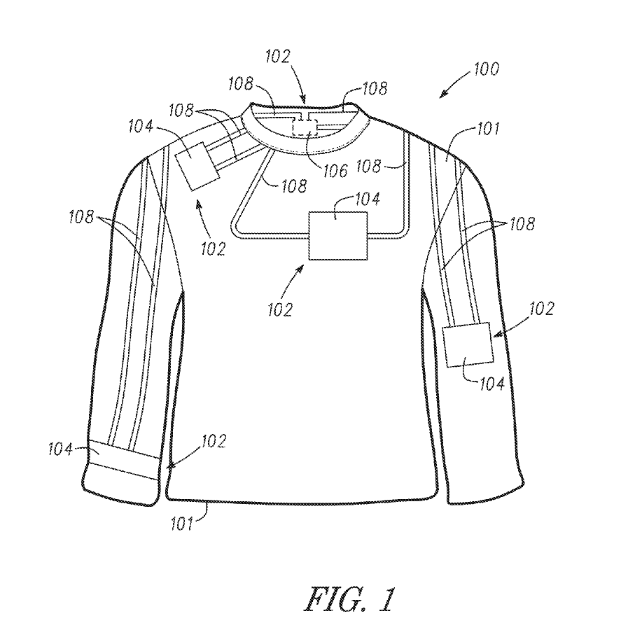

FIG. 1 shows one example of a garment 100. As shown in FIG. 1, the garment 100 includes a clothing article, for instance a shirt. In another example, the garment 100 (as described herein) includes, but is not limited to, one or more of a garment such as a vest, shirt, pant or the like. In another example, the garment 100 includes, but is not limited to, a sleeve, cuff or the like configured for reception on one or more limbs or portions of the anatomy (such as the torso) of the user. In another example, the garment 100 includes a jewelry article, strap or the like configured for buckling to one or more of the limbs or the body of the user. As shown in FIG. 1, the garment 100 includes a garment substrate 101. The garment substrate 101 includes a deformable substrate, such as a textile. In another example the garment substrate 101 includes one or more elastomers in combination with one or more of textile or elastomers forming the remainder of the garment 100.

As further shown in FIG. 1, the garment 100 includes a physiological characteristic measurement system 102. The physiological characteristic measurement 102 system includes a one or more sensor assemblies 104 (as shown, a plurality) distributed around the garment 100. As will be described herein, the sensor assemblies 104 in combination with a controller 106 are configured to measure physiological movements at one or more of the limbs, organs or the like of the user. The values of the measurements corresponding to the movements at the limbs or one or more of the organs of the user are interpreted at the controller 106 to accordingly facilitate identification and assessment of one or more physiological characteristics of the user of the garment 100. For instance as shown in FIG. 1, one or more of the sensor assemblies 104 are provided on the sleeves of the garment 100. Another sensor assembly 104 is provided at a shoulder portion of the garment 100. The sensors 104 provided at the limbs and in this example the shoulder are configured for measuring one or more movements of the limbs or upper torso of the user. In one example, described herein, the controller 106 interprets measurements from each of the sensor assemblies 104 and by way of integration of comparisons of the measurements at each of the sensor assemblies 104 is able to do one or more of identification or assessment (e.g., determine magnitude, vector or the like) of a gesture or physiological movement of the user. In another example, the physiological character measurement system 102 for instance in controller 106 is able to identify each of a gesture, physiological characteristic, corresponding vectors, magnitudes or the like based on sensed measurements from each of the sensory assemblies 104.

As described herein, the physiological character measurement system 102 includes one or more sensor assemblies 104 provided on the garment 100. In another example, the sensor assemblies 104 are provided on patches configured for attachment to the skin of the user (e.g., by adhesives, straps, buckles or the like). The sensor assemblies 104 are in one example provided at various locations on the garment 100. As previously described herein, at least some of the sensor assemblies 104 are provided on the limbs for instance the arms (or legs) of the garment 100 as well as one or more shoulder portions of the garment 100. In another example, one or more sensor assemblies 104 are provided adjacent to one or more locations corresponding to the organs of the user. For instance, the sensor assembly 104 provided on the chest of the garment 100 is positioned near to the lungs and heart of the user when wearing the garment 100. As described herein the sensor assemblies 104 are configured for measurement of physiological characteristics including movements of the user and physiological organ characteristics for instance respiration characteristics, heartbeat characteristics or the like.

Referring again to FIG. 1, the sensor assemblies 104 are in one example interconnected with the controller 106 by one or more interconnecting conductive traces 108. As shown in FIG. 1, in the example garment 100 the interconnecting conductive traces 108 extend from each of the sensor assemblies 104 toward the controller 106 provided at the back portion of the garment 100. As shown in FIG. 1, the controller 106 is similarly sized to the tag of a garment and is in one example positioned in place of or within the tag of the garment at the collar. In other examples, the controller 106 is coupled with another portion of the garment 100 including, but not limited to, a collar, a cuff, a sleeve, a seam, the chest, back, should or the like. Ins till other examples, the controller 106 is removably coupled with the garment (e.g., to facilitate replacement, washing of the garment or the like).

In operation, each of the sensor assemblies 104 (where a plurality of sensor assemblies are provided) measures deformation at a location corresponding to the location of the sensor assembly 104 on the garment 100. The measured deformation corresponds to movement of the user of the garment 100 including for instance physiological movement of the limbs, torso or the like. In another example, the physiological movement corresponds to one or more body functions including lung respiration, heartbeats or the like. As described herein, in one example one or more of the sensor assemblies 104 includes one or more sensor elements configured to measure the physiological movement. Optionally, each of the sensor assemblies 104 include a plurality of sensor elements configured to measure the physiological movement at the corresponding location along one or more axes to provide a composite of the physiological movement to facilitate the determination of one or more of a vector or magnitude for the movement.

The sensor assemblies 104 include the sensor elements therein. In one example, the sensor elements include piezo elements (e.g., piezo resistive or piezoelectric elements) configured to alter characteristics (e.g., resistance, voltage or the like) with corresponding movement of the sensor assemblies 104. That is to say, with deformation of the sensor assemblies 104 (e.g., a deformable sensor assembly positioned on a deformable substrate) the piezo elements deform and accordingly their characteristics (resistance or voltage generated) change in a measurable manner The controller 106 uses the value of the deformation from the sensor assemblies 104 to determine a value for the corresponding movement at each of the sensor assemblies 104. The controller 106 identifies the movement based on the interpretation of measurements from one or more of the sensor assemblies 104. In an example, where the sensor assembly 104 includes a plurality of sensor elements, for instance two or more piezo elements positioned in differing orientations, multiple components of the measured movement are conveyed to the controller 106. The controller 106 uses the multiple components to determine a vector including magnitude and direction of the movement at the location of the sensor assembly 104.

In another example, where the physiological character measurement system 102 includes a plurality of sensor assemblies 104 additional measurements including movement at each of the sensor assemblies 104 is consolidated at the controller 106, compared against thresholds, and the comparisons are used to identify the type of the motion or physiological characteristic and in some examples its one or more of its magnitude or vector. By analyzing the measurements at each of the sensor assemblies 104 (comparing to thresholds) and evaluating each of the measurements together accurate and detailed identification of physiological movement including human gestures, physiological organ characteristics or the like is realized.

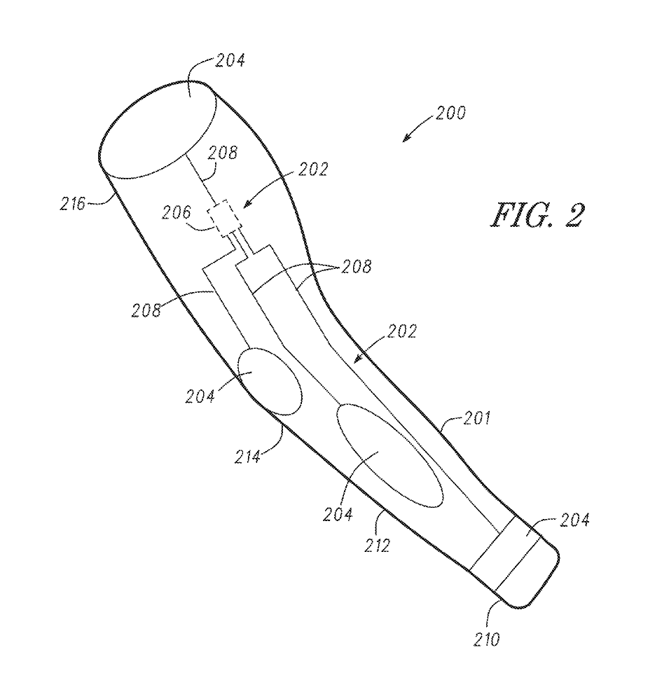

FIG. 2 shows another example of a garment 200. As shown, the garment 200 includes one or more of a cuff or a sleeve configured for reception around one or more limbs of the user, in this example the right arm. In another example, the garment 200 includes, but is not limited to, one or more of multiple cuffs, sleeves or the like positioned at various locations along the limb for instance at the shoulder, the elbow, the forearm and the wrist. In yet another example, the garment 200 includes a plurality of straps, buckles, adhesive patches or the like that allow for positioning of a plurality of sensor assemblies such as the sensor assemblies 204 shown in FIG. 2 at a corresponding plurality of locations along the limb.

As further shown in FIG. 2, the garment 200 includes a physiological character measurement system 202. The physiological character measurement system 202 in FIG. 2 is similar in at least some regards to the physiological character measurement system 102 shown in FIG. 1. The system 202 includes a plurality of sensor assemblies 204 provided at one or more locations along the garment 200. For instance, sensor assemblies 204 are provided at one or more of the wrist 210, forearm 212, elbow 214 and the shoulder 216. In another example, the garment 200 includes one or more sensor assemblies 204 provided at one or more of the locations previously described or differing locations of the limb. As previously described herein, the provision of a plurality of sensor assemblies 204 at various locations along the garment 200 accounts for additional measurement of movement of the limb and corresponding enhanced identification of physiological characteristics including gestures, their magnitude, vectors or the like.

Referring again to FIG. 2, the physiological character measurement system 202 includes the plurality of sensor assemblies 204 at the various positions along the garment 200. The sensor assemblies 204 are interconnected with a controller 206 (in this example) positioned on the garment 200. The interconnections are provided in one example by interconnecting conductive traces 208 extending from each of the sensor assemblies 204 to the controller 206.

The garment 200 including the physiological character measurement system 202 is configured for measurement of one or more physiological characteristics according to deformation of sensor elements in each of the sensor assemblies 204. For instance, as the user of the garment 200 moves the corresponding limb one or more of the sensor assemblies 204 for including a deformable substrate is deformed by the movement. For instance, the underlying textile (e.g., a deformable substrate) of the garment 200 is deformed by the movement of the limb which is measured by the sensor elements in the sensor assemblies 204. In another example, each of the sensor assemblies 204 includes an elastic base or an elastic substrate (another example of a deformable substrate) coupled with garment 200 and the deformation of the garment is transmitted into the elastomer substrate to the sensors of the sensor assembly 204 to facilitate the measurement of the physiological characteristic of the movement of the limb within the garment 200. By providing a plurality of sensor assemblies 204, each sensor assembly 204 having one or more sensor elements therein, measurement of the movement of the limb by the physiological character measurement system 202 is enhanced and allows for improved identification of physiological movement including identification of gestures, magnitude and vectors of the same.

For instance, gestures of the hand including movement of the hand by way of flexure at the wrist is measured in one example by the sensor assemblies 204 provided at least at the wrist 210 and the forearm 212. The detected movement at each of the sensor assemblies 204 at the wrist 210 and the forearm 212 is interpreted by the controller 206 for instance by way of comparison with a plurality of thresholds for each of the sensor assemblies 204 to facilitate the identification of a gesture and in one or more examples its vector or magnitude. Similarly, the bending of the arm is measured in one example by the sensor assembly 204 associated with the elbow 214. Deformation of the garment 200 and the corresponding sensor elements of the sensor assembly 204 at the elbow 214 facilitate the measurement of the degree of bend in the arm and accordingly allows for identification of a gesture such as bending of the arm during a throwing motion and the degree of its bending. In another similar manner, the sensor assembly 204 at the shoulder 216 in one example includes one or more sensor elements configured to measure the rotation of the arm, its flexure (e.g., lateral extension from the body), extension of the arm anteriorly or posteriorly or the like. The controller 206 interprets the information from the sensor assembly 204 at the shoulder 216 to identify that portion of the gesture corresponding to the movement of the shoulder 216 (e.g., rotation of the shoulder, lateral movement, anterior movement, posterior movement and the like).

In yet another example, the controller 206 interprets measurements from each of the sensor assemblies 204 corresponding to physiological characteristics at each of the locations on the garment 200 having a sensor assembly 204 thereon. For instance, movement is measured at each of the wrist 210, forearm 212, elbow 214 and shoulder 216 by the corresponding sensor assemblies 204. A combination of movements measured at each of the sensor assemblies 204 facilitates the identification of complex movements of the limb including for instance waving, throwing motions, interactions with virtual screens, swinging motions, raising or lowering gestures, directional motions with the arm or the like. Additionally, the controller 206 is able, by way of interpretation of the measurements from each of the sensor assemblies 204 and comparison of the measurements to one or more thresholds, to assign one or more of a magnitude, vector or the like to each of the movements or a composite movement of the entire limb.

The controller 206 is equipped in one example with a transmitter or wired connection to facilitate the communication of the information including the identified physiological characteristic (a gesture), its magnitude, vector or the like to one or more systems including an interaction system that converts the measured motion of the garment 200 and the corresponding limb to instructions for one or more systems including, but not limited to, machines, visual and audio systems, game systems, systems for observation and documenting of the physiological movements of the wearer for healthcare purposes or the like. Further, although the garment 200 shown in FIG. 2 is in the shape of a sleeve and configured for reception of an arm, the garment 200 and the sensor assemblies 204 are similarly employed in other profiles including, but not limited to, one or more of a shirt, coat, vest, suit, sleeve, pant, glove including one or more digits, sock, shoe, or the like.

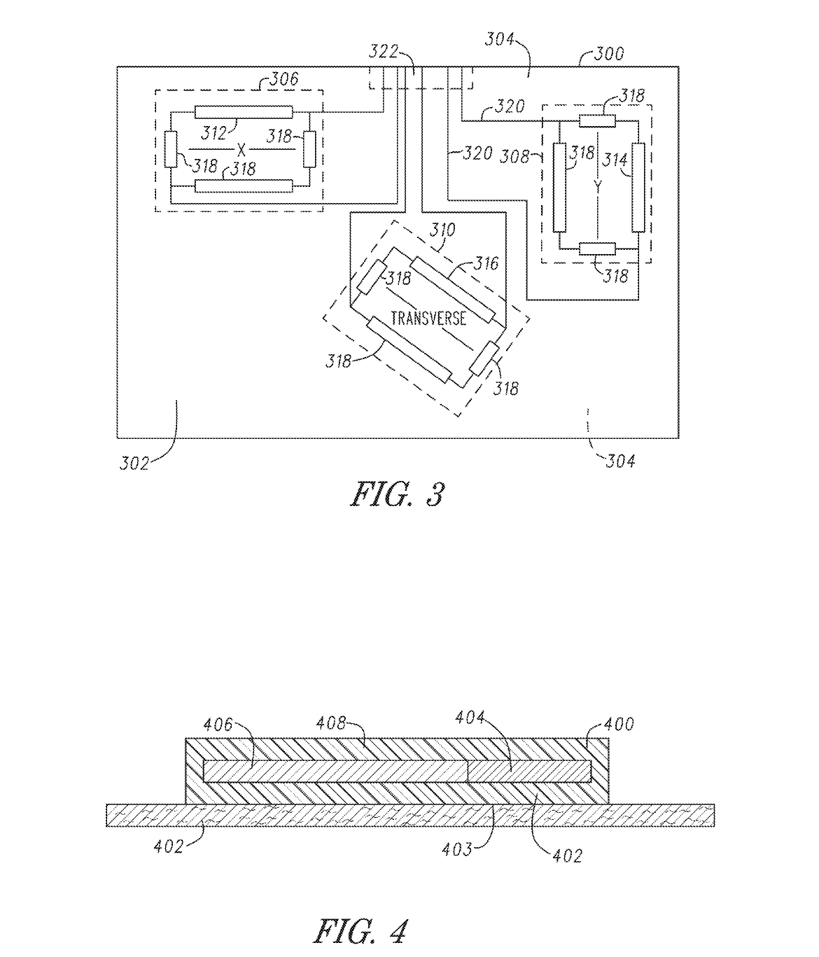

FIG. 3 shows one (schematic) example of a sensor assembly 300. The sensor assembly 300 corresponds in at least some examples to one or more of the sensor assemblies 104, 204 previously described herein. As shown in FIG. 3, the sensor assembly 300 includes a plurality of sensor elements for instance first, second and third sensor elements 306, 308, 310. In another example the sensor assembly 300 (as well as the sensor assemblies 104, 204) includes one or more sensor elements.

The sensor assembly 300 shown in FIG. 3 includes a deformable substrate 302 for coupling each of the sensor elements 306-310. As shown the deformable substrate 302 extends across the sensor assembly 300 and includes a body side interface 304. In one example, the body side interface 304 is configured for contact with the skin of the user, includes an adhesive for contact with the skin or includes an adhesive or other interface configured for coupling with a garment such as the garments 100, 200 shown in FIGS. 1 and 2. In another example, the deformable substrate 302 includes a textile, for instance a portion of the garment 100 or the garment 200 shown in FIG. 1 or 2, respectively. That is to say, the sensor assembly 300 is incorporated with the garment.

In another example, the deformable substrate includes an elastomeric material (an elastomer substrate) that is configured to deform with deformation of an underlying textile or the skin of the user during physiological movement (e.g., motion of the limbs, physiological organ movement such as respiration, heartbeats or the like). The deformation of the deformable substrate 302 is conveyed to the sensor elements, and corresponding deformation of the sensor elements 306-310 (e.g., portions of the elements such as piezo elements) is measured. Where the deformable substrate 302 includes an elastomer, the elastomer includes, but is not limited to, one or more of thermoplastic polyurethane, polydimethylsiloxane, silicone elastomers, butyl rubber or the like. One or more of these materials provides an elastomeric substrate that readily conveys deformation of the underlying material (the garment or skin) to each of the sensor elements provided with the sensor assembly 300.

Referring again to FIG. 3, as shown the sensor assembly 300 includes one or more sensor elements 306, 308, 310. As previously described herein, each of the sensor elements 306-10 includes in one example at least one piezo element, such as first, second and third piezo elements 312, 314, 316. Optionally, the piezo elements 312, 314, 316 are arranged with one or more supplemental components, for instance resistor elements 318 to form a plurality of Wheatstone bridges for the sensor assembly 300. As shown in FIG. 3, each of the sensor elements 306, 308, 310 are bracketed by dashed lines. The components found within the sensor element 306 (in one example) including the first piezo element 312 and the optional resistor elements 318 form the sensor element (and similarly the other sensor elements 308, 310).

As will be described in further detail herein, in one example the piezo element 312 is formed with a cured piezo ink provided on the deformable substrate 302. Similarly, the substrate conductive traces 320 formed between the resister elements 318, the piezo elements 312, 314, 316 and the like as well as traces extending to a sensor assembly interface 322 are formed with one or more cured conductive inks applied to the deformable substrate 302. One or more of the piezo elements 312, 314, 316 and the conductive traces 320 (and other conductive traces herein) are formed with other methods including, but not limited to, sputtering of a piezo material followed by an annealing process such as laser annealing, the coupling of piezo films to the substrate 302 (e.g., the garment or elastomer) through lamination, or the like. In another example, one or more of piezo cables or organic piezo strands (e.g., polyvinylidene fluoride-class polymers) are laminated or woven into the deformable substrate 302.

Referring again to FIG. 3, in the example shown the sensor assembly 300 includes a plurality of sensor elements such as the first and second sensor elements 306, 308. As shown, each of the piezo elements 312, 314 are provided in differing orientations. For instance, the first piezo element 312 is aligned with an x axis within the first sensor element 306. Similarly, the second piezo element 314 is aligned with a y axis within the second sensor element 308. Including a plurality of sensor elements positioned in differing orientations on the sensor assembly 300 allows for the measurement of components of the physiological movement at the location of the sensor assembly 300. In one example, referring in part to FIG. 2, where one or more sensor elements such as the first and second sensor elements 306, 308 are provided at a multi-directional joint such as the shoulder 216, the plurality of orientations of the piezo elements 312, 314 allow for component measurement of the shoulder movement. By measuring the component movement an overall composite movement is accordingly generated at the controller 206 by blending of the measurements of each of the first and second piezo elements 312, 314 of the first and second sensor elements 306, 308. Additionally, by measuring the components of the movement, direction and magnitude are in one example determined (e.g., with the controller 206 by way of sine, cosine, tangent conventions or the like).

As further shown in FIG. 3, in one example the sensor assembly 300 includes a third piezo element 316 incorporated with a third sensor element 310. The third piezo element 316 is provided in a transverse orientation relative to each of the first and second piezo elements 312, 314. The third sensor element 310 optionally includes resistor elements 318 arranged to form a Wheatstone bridge in a similar manner to the first and second sensor elements 306, 308. In one example, the measurement of deformation at the third piezo element 316 is used in combination with the measurements of deformation of each of the first and second piezo elements 312, 314 to filter noise. In one example, noise generated by one or more of the garments 100, 200 (e.g., by wrinkling, rustling of the fabric, relative movement of the fabric of the garment 100, 200 relative to the skin of the user or the like) is measured with each of the first, second and third sensor elements 306, 308, 310. The noise is filtered by way of comparison of the measurements of the third sensor element 310 to the component measurements of each of the first and second sensor elements 306, 308 or their composite vector value determined at the controller 206.

As further shown in FIG. 3, the sensor assembly 300 includes a sensor assembly interface 322. In one example, where the sensor assembly 300 is provided as a component for assembly with a garment 100, 200 the sensor assembly interface 322 provides the interface for one or more interconnecting conductive traces 108, 208 (as shown in FIGS. 1 and 2). That is to say, the substrate conductive traces 320 extend from each of the sensor elements 306, 308, 310 to the sensor assembly interface 322 for connection with the interconnecting conductive traces 208 of the garments 100, 200.

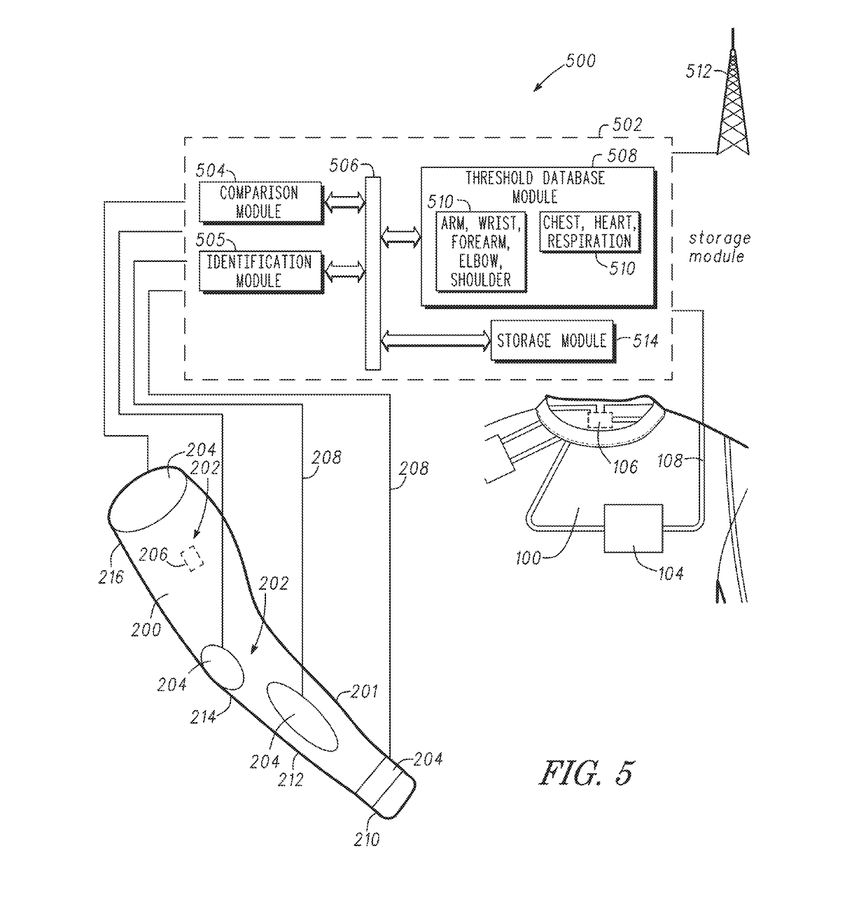

FIG. 4 shows another example of a sensor assembly 400 in cross section. The sensor assembly 400 is similar in at least some regards to the views of the sensor assemblies 104, 204, 300 shown in FIGS. 1, 2 and 3, respectively. For instance, the sensor assembly 400 includes one or more sensor elements including a piezo element 404 and a plurality of substrate conductive traces 406 provided on a deformable substrate 402. As previously described, the sensor element is in one example a piezo element 404 configured to measure movement of a limb or organ by way of deformation of the piezo element 404 (e.g., through deformation of the substrate including an elastomer, textile or the like). In one example, the piezo element 404 includes a piezo resistive element that changes resistance in a graduated manner corresponding to the degree of deformation of the piezo element 404. In another example, the piezo element 404 includes a piezo-electric element configured to generate electricity (e.g., voltage, potential, current or the like) in a graduated manner corresponding to the degree of deformation of the element 404. The substrate conductive traces 406 are provided and in communication with piezo element 404 to convey the measureable changes in resistance (or voltage of a piezoelectric element) from the sensor assembly 400 to one or more control systems including for instance the controllers 106, 206 shown in FIGS. 1 and 2, respectively.

As further shown in FIG. 4, in one example the deformable substrate 402 is provided with a body side interface 403. The body side interface 403 includes an adhesive or other coupling feature configured to couple the sensor assembly 400 to one or more of a garment, skin or the like. In the example shown in FIG. 4, the body side interface 403 is coupled with an underlying textile 402. In another example, the piezo element 404 and the substrate conductive traces 406 are directly coupled with the textile 402. The textile 402 thereby serves as the deformable substrate of the sensor assembly.

In another example, the sensor assembly 400 includes an encapsulant 408 extending around the components of the sensor assembly 400 including the substrate conductive traces 406 and the one or more piezo elements 404 housed therein. The encapsulant 408 provides a protective cover to the components of the sensor assembly 400. Optionally, the encapsulant is deformably and facilitates the deflection or deformation of the one or more piezo elements 404 to ensure measurement of deformation of a garment such as the garments 100, 200, the skin of the user or the like. As previously described herein, the deformation of either of the garments 100, 200 or the skin corresponds to movement of the user at one or more joints, limbs or the like. In one example, the deformable substrate 402 (and optionally the encapsulant 408) includes an elastomer. The elastomer includes one or more of a thermoplastic polyurethane, polydimethylsiloxane, silicone elastomers, butyl rubbers or the like. The deformable substrate 402 as well as the encapsulant 408 provides a protective envelope to the components of the sensor assembly 400 including the piezo elements 404 and the substrate conductive traces 406. As shown in FIG. 3, a sensor assembly interface 322 is provided with the sensor assembly (including the assembly 400) facilitate coupling with conductive traces and other components of a system including other sensor assemblies 300 and controllers (e.g., controllers 106, 206). Optionally, the sensor assembly interface 322 is a gap or recess formed in the encapsulant 408 to facilitate the coupling. In another example, the sensor assembly interface 322 includes a fitting having one or more contacts, ports, plugs or the like configured for coupling with conductive traces.

Additionally, the deformable substrate 402 provides a relatively planar feature, such as the body side interface 403 that is readily coupled to an underlying material such as the textile 402 or skin. In one example, the deformable substrate 402 is readily attached to a textile 402 through the application of heat (e.g., by ironing of the sensor assembly 400 over the textile 402 at a desired location on a garment). In another example, the body side interface 403 includes an adhesive applied immediately before coupling with the textile 402 or applied to the deformable substrate 402 at manufacturing and exposed by way of peeling a removable liner.

The sensor assembly 400 is constructed with one or more methods as described herein. The deformable substrate 402, in one example an elastomer, is formed by molding of elastomeric material into a desired shape. In other examples, the deformable substrate 402 is formed by one or more of sputtering the elastomeric material, cutting a lineal elastomer sheet into the desired shape for the deformable substrate 402 or the like.

Each of the piezo elements 404 and the substrate conductive traces 406 are formed on the deformable substrate 402 in one or more methods including the application and curing of piezo inks and conductive inks on the deformable substrate. In one example a piezo ink is applied to the deformable substrate 402 by way of one or more of stenciling, screen printing, sputtering or patterning of the piezo elements 404 onto the deformable substrate 402. In an example including patterning, the piezo element 404 is applied by way of masking the deformable substrate 402 and using an etchant to etch the one or more desired piezo elements 404 onto the deformable substrate. In another example, the piezo ink once applied to the deformable substrate 402 is cured, for instance by way of allowing the ink to set over time, applying heat to the sensor assembly 400 to cure the ink, baking the sensor assembly 400 or ironing the sensor assembly 400 to accordingly set the piezo ink.

In a similar manner, the substrate conductive traces 406 (and optionally the interconnecting conductive traces 108, 208 shown in FIGS. 1 and 2) are formed in the sensor assembly 400 (or in the garments 100, 200) by way of the application of a conductive ink. The conductive ink is optionally applied to the deformable substrate 402 in a similar manner to the examples provided for the piezo ink. The conductive ink is applied by one or more of stencil printing, screen printing, jet printing or the like. Other methods used to deposit conductors enabling connectivity of the system include, but are not limited to sputtering of conductive materials, conductive organic or metallic materials in threads or strands woven into the deformable substrate 402 (textile or elastomer), conductive traces 406 (with a mask and etching) or the like. The applied conductive ink is cured on the deformable substrate 402. Curing is in one example conducted in a similar manner to the piezo ink as previously discussed herein. For instance the conductive ink is allowed to set for a specified period of time, or heat is applied through one or more of baking, ironing or the like.

Optionally, after the substrate conductive traces 406 and the one or more piezo elements 404 are set (cured) on the deformable substrate 402 the encapsulant 408 is applied over top of the piezo elements 404 and substrate conductive traces 406. In one example, the encapsulant 408 includes materials similar to the elastomer of the deformable substrate 402. In another example, the material of the encapsulant 408 is applied by molding, sputtering or the like over top of the components of the sensor assembly 400. In an example, where the encapsulant 408 and the deformable substrate 402 include similar elastomers the elastomer of the encapsulant 408 readily bonds with the deformable substrate 402 to seal the substrate conductive traces 406 and the piezo elements 404 therein. In another example, a lineal sheet of the encapsulant 408 (for instance constructed with the same or similar material as the deformable substrate 402) is adhered to and applied over the components of the sensor assembly 400. The adhered sheet is coupled with the deformable substrate 402 to form a laminate including the substrate conductive traces 406 and piezo element 404 between the encapsulant 408 and the deformable substrate 402.

In still another example, an adhesive or other coupling feature is applied to the body side interface 403 to ensure ready coupling of the sensor assembly 400 to either or both of the skin of the user, a garment, cuff, sleeve, jewelry article or the like. Optionally, the sensor assembly 400 is incorporated with a jewelry element including, but not limited to, a bracelet, sleeve or the like configured for coupling around a limb, torso or the like.

FIG. 5 shows one example of a physiological character measurement system 500 that is similar in at least some regards to the systems 102, 202 previously described herein. In one example, the physiological character measurement system 500 includes one or more of the garments 100, 200 previously shown in FIGS. 1 and 2. The physiological character measurement system 500 is not limited to either of these garments 100, 200. For instance, as previously described one or more sensor assemblies 104, 204 shown in FIGS. 1, 2 or the sensor assemblies 300, 400 shown in FIGS. 3 and 4 are included in one or more components (garments) for instance straps, adhesive patches, sleeves, garments, jewelry or the like configured for coupling with the user. That is to say, the sensor assemblies 104, 204 shown in FIG. 5 are not limited for instance to the garments 100, 200 shown therein.

Referring again to FIG. 5, the sensor assemblies 104 are associated with corresponding garments 100, 200. In another example, the garments 100, 200 are a unitary garment (e.g., shirt, pant, suit or the like) including the sensor assemblies 204, 104 provided thereon. In the example shown, the sensor assembly 104 is in a position corresponding to a location of one or more organs of the user such as the lungs or heart. As previously described herein, the sensor assembly 104 is optionally configured to measure movement of the organs of the user (such movement mirrored in the deformation of the deformable substrate and the one or more sensor elements such as piezo elements in the sensor assembly). Deformation of the sensor elements is assessed by the controller such as the controller 502 to identify one or more physiological characteristics including for instance heart rate, respiration rate or the like.

As also shown in FIG. 5, the garment 200 includes a plurality of sensor assemblies 204 provided at various locations along the garment 200. For instance, sensor assemblies 204 are provided at each of the wrist 210, forearm 212, elbow 214 and shoulder 216. In another example, the one or more sensor assemblies 204 are provided at one or more of these corresponding locations or other locations on the garment 200.

Each of the sensor assemblies 104, 204 are coupled with the controller 502 by way of interconnecting conductive traces 108, 208. As previously described and shown in FIGS. 1 and 2, the interconnecting conductive traces 108, 208 allow for communication between the sensor assemblies 104, 204 and the controller 502. As described herein, the controller 502 with measured values of movement from each of the sensor assemblies 104, 204 is configured to identify one or more physiological characteristics based on the measured values from each of the sensor assemblies. For instance, by assessing a plurality of measured values (including magnitude, vectors or the like) from a plurality of locations the controller 502 identifies one or more physiological characteristics (gestures, organ functions or the like), magnitude, vector of a gesture or movement or the like.

Referring now to the controller 502, as shown the exemplary controller includes one or more modules configured to identify one or more movements associated with physiological characteristics measured by each of the sensor assemblies 104, 204. In the example shown, the controller 502 includes a comparison module 504. The comparison module 504 is configured to compare measured values of physiological movement corresponding to deformation of the sensor elements (e.g., piezo elements) at each of the sensor assemblies 204. By comparison of the measurements at each of the sensor assemblies 104, 204 to corresponding thresholds the identification module 505 is able to identify the physiological characteristic (movement, organ function or the like) and in some examples identify the vector or magnitude of the physiological characteristic. As further shown in FIG. 5, the comparison module 504 is in communication by way of an interface 506 (such as a bus) with the identification module 505. As previously described, the identification module 505 uses the comparisons conducted at the comparison module 504 to assess physiological movement (measurement of physiological movement through deformation of one or more of the sensor elements at the sensor assemblies) to identify a physiological characteristic and at least in some examples one or more of its vector or magnitude.

As further shown in FIG. 5, in one example the controller 502 includes a threshold database module 508. The threshold database module 508 is in communication with at least the comparison module 504 by way of the interface 506. The threshold database module 508 includes one or more thresholds used by the comparison module 504 for comparison with measurements taken at one or more of the sensor assemblies 104, 204. In one example, the threshold database module 508 includes at least one threshold for each of the one or more sensor assemblies 104, 204. For instance, with the single threshold for one or more of the sensor assemblies the comparison module 504 is configured to compare physiological movement sensed at one or more of the sensor assemblies 104, 204 and if the compared physiological movement is greater than or less than the respective threshold the identification module 505 identifies movement of the limb or organ by way of exceeding or not meeting the threshold (or thresholds at a plurality of sensor assemblies).

In another example, the threshold database module 508 includes a plurality of thresholds in physiology modules 510. For instance, one or more physiology modules 510 are provided for the arm and chest as shown in FIG. 5. In such an example, with the arm physiology module 510, one or more thresholds are provided for each of the associated sensor assemblies 204 provided at one or more of the wrist 210, forearm 212, elbow 214 and shoulder 216 of the garment 200. Optionally, a plurality of thresholds are provided for each of these locations along the garment 200. By providing a plurality of thresholds for each of the locations of the sensory assemblies 204 enhanced discrimination and identification of physiological movement at each of the locations is provided. For instance, the measured value at each of the sensor assemblies 204 is compared at the comparison module 504 with a plurality of corresponding thresholds for each of the locations. Accordingly, the identification module 505 is thereby able to identify a degree of motion at each of the locations to more accurately identify a gesture or movement of the limb (as well as magnitude or vector) housed within the garment 200.

In another example, for instance where one or more of the sensor assemblies 104, 204 includes a plurality of sensor elements (as shown in FIG. 3) including at least first and second sensor elements 306, 308, and in some examples a third sensor element 310, the comparison module 504 in cooperation with the identification module 505 identifies the direction of motion at each of the locations of the sensor assemblies 104, 204. For instance, as previously described herein and shown in FIG. 3, in one example a first sensor element 306 is provided along an x axis while a second sensor element 308 is provided along a differing axis, for instance a y axis. In one example, the identification module 505 interprets the movement at each of the first and second sensor elements 306, 308 (including for instance first and second piezo elements 312, 314 respectively) to determine components of an overall composite movement at one or more of the locations such as the locations (e.g., the wrist 210, forearm 212, elbow 214 or shoulder 216). The refined identification provided by the identification module 505 allows for the assignment of at least a magnitude or a vector (with two or more sensors elements) at each of the locations.

Accordingly, the controller 502 accurately identifies with high resolution physiological movement of the user. For instance, in the example shown in FIG. 5 the controller 502 including the comparison module 504 and the identification module 505 is configured to measure movement including the magnitude (by way of a plurality of thresholds) and the direction of movement (for instance with first and second sensor elements 306, 308). By measuring each of the magnitude and direction of motion at one or more locations on the garment 200 for instance at the wrist 210, forearm 212, elbow 214 and shoulder 216 the physiological character measurement system 500 accurately and with high resolution identifies gestures including, but not limited to, gestures of the arm, the hand, and other limbs or digits of limbs through these comparisons and assessment.

One example of a complex movement of a limb such as the arm measured and identified by the physiological character measurement system 500 is provided herein. In one example, the complex motion measured and identified in this example includes motion of the arm in an expanding fashion for instance with the arm and right hand extending from the left side of the torso in an outward and diagonally upward manner, for instance away from the body of the user toward an upward location with the hand extending away from the user (e.g., a sweeping motion from the lower left of the user toward a location above the right shoulder). In such an example, the complex motion generates corresponding deformation at one or more of the sensor assemblies 204 provided on the garment 200. For instance, the sensor assembly 204 at the shoulder 216 measures motion of the shoulder in a rotating fashion for instance in a crossing fashion across the torso of the user from the hip in the direction of the right shoulder and away from the body. Similarly, the elbow 214 experiences a flexing motion in the form of a contraction at the sensor assembly 204 at the elbow 214. In another example, at least the wrist 210 including the sensor assembly 204 registers rotation of the wrist into an outward waving fashion away from the body. Optionally, rotation of the forearm 212 in a clockwise fashion (along the axis of the forearm) is detected at the sensor assembly 204 associated with the forearm 212. Each of the movements measured at each of the sensor assemblies 204 for the respective locations on the garment 200 are assessed at the comparison module 504, for instance against a plurality of thresholds for each of the locations such as locations 210, 212, 214, 216. As previously described, where a plurality of thresholds are provided for each of these locations at the threshold database module the comparison module 504 is configured to compare the measurements at each of these locations to the plurality of thresholds.

The corresponding comparisons are forwarded on to the identification module 505 and the module 50t interprets the comparisons to thereby blend the measurements and determine the corresponding motion of the limb within the garment 200. That is to say the measured physiological movement (deformation of the sensor elements such as first and second piezo elements 312, 314) of each of the sensor assemblies 204 is synthesized and then identified by the identification module 505 as an overall movement of the arm for instance in an sweeping fashion beginning at the left hip of the user and expanding outwardly past the right shoulder of the user with rotation of the hand (as measured with one or more of the forearm 212 or wrist 210 sensor assembly 204). One or more of magnitude and a vector for the motion at each of these locations 210-216 is determined by way of comparison of the physiological movement to a plurality of thresholds at each of the locations and in some examples by the inclusion of a plurality of sensor elements 306, 308 (including corresponding piezo elements) to generate vectors. Accordingly, by way of synthesizing the outputs of a plurality of sensor assemblies 204 at a plurality of locations the identification module 505 of the physiological character measurement system 500 accurately identifies physiological movement, its magnitude and direction (e.g., vector) with greater resolution relative to previous systems.

Referring again to FIG. 5, in one example the sensor assembly 104 is provided with the physiological character measurement system 500 at a location corresponding to one or more organs of the user, for instance organs that generate physiological movement within the user such as the lungs, heart or the like. As shown in FIG. 5, the sensor assembly 104 is provided (on the exemplary garment 100) at the upper torso of the user overlying one or more of the heart or the lungs. As previously described herein, the sensory assembly 104 is configured to measure deformation at the location with one or more sensor elements such as the first and second sensor elements 306, 308 shown in FIG. 3. The measured deformation corresponds to one or more of respiration, heart rate (by way of measured heart beats) or the like.

As previously described herein, the threshold database module 508 in one example includes a physiology module corresponding to one or more of a heartbeat threshold (or thresholds) or respiration threshold (or thresholds). The comparison module 504 in such an example compares the measured deformation at the sensor assembly 104 with one or more thresholds from the physiology module 510. The identification module 505 assesses the comparison of the measurements to the relevant thresholds and accordingly identifies one or more of a user's heart rate, respiration rate or the like (e.g., by way of counting measurements that meet or exceed the specified thresholds).

In another example, the physiological characteristics measurement system 500, for instance the controller 502 includes a storage module 504. In one example, the storage module 504 stores one or more physiological characteristic measurements, identified physiological characteristics including, but not limited to, one or more of movements limbs (gestures, magnitudes, vectors or the like) or physiological characteristics such as heart rate, respiration rate or the like over a period of time. Optionally, the controller 502 includes a transmitter 512 configured to convey stored information or ongoing measurements from the controller 502 to one or more other systems including for instance a PC, tablet computer, PDA, smartphone, game console, interactive monitor or the like. In still another example, the transmitter 512 includes a transceiver figured to transmit and receive data including, but not limited to, calibration data, updated thresholds or the like. In still another example, the controller 502 is provided as an onboard component for instance an onboard component of one or more of the garments 100, 200 shown in FIGS. 1 and 2. For instance, the controller 502 is provided in a similar manner to the controller 206 in FIG. 2 and the controller 106 in FIG. 1. That is to say, the controller 502 is a compact controller provided in a tag or adhesive patch provided on the garment 100, 200. In another example, the controller 502 is provided in a removable manner, for instance with an interfacing socket to couple with one or more interconnecting conductive traces 108, 208 of the garments 100, 200. The controller 502 is removable to facilitate washing of one or more of the garments 100, 200, replacement or the like. Optionally, the controller 502 (or any of the controllers 106, 206) is a textile integrated controller. For instance, the controller is a deformable electronic system (e.g., operational with stretching, folding or the like). In another example, the deformable controller 502 (or one or more of controllers 106, 206) includes processing and connectivity (or wireless) components permanently integrated into a garment, such as garments 100, 200.

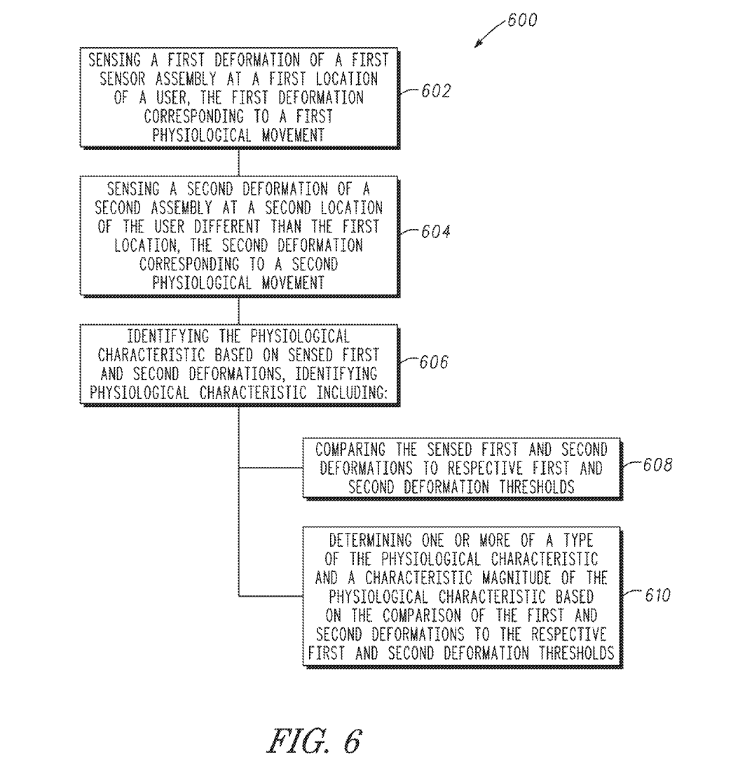

FIG. 6 shows one example of a method 600 for identifying a physiological characteristic. In describing the method 600, reference is made to one or more components, features, functions and steps previously described herein. Where convenient reference is made to the components, features, steps and the like with reference numerals. Reference numerals provided are exemplary and are nonexclusive. For instance components, features, functions, steps and the like described in the method 600 include, but are not limited to, the corresponding numbered elements provided herein, other corresponding features described herein (both numbered and unnumbered) as well as their equivalents.

At 602, the method 600 includes sensing a first deformation of a first sensor assembly 204 (or 104) at a first location of a user such as a first body location (e.g., location on a limb, digit, body part or the like). The first deformation corresponds to a first physiological movement. At 604, a second deformation of a second sensor assembly 204 (or 104) is sensed at a second location of a user such as a second body location different than the first location. The second deformation corresponds to a second physiological movement at that second location.

At 606, identifying the physiological characteristic is conducted based on the sensed first and second deformations. Identifying the physiological characteristic includes at 608 comparing the sensed first and second deformations to respective first and second deformation thresholds (e.g., thresholds provided in a threshold database module 508 as shown in FIG. 5). In another example, comparing the sensed first and second deformations includes comparing the sensed first and second deformations to a plurality of thresholds stored at the threshold database module 508.

At 610, identifying the physiological characteristic includes determining one or more of a type of the physiological characteristic or characteristic magnitude (including a vector) of the physiological characteristic based on comparisons of the first and second deformations to the respective first and second deformation thresholds. That is to say, in at least one example a physiological characteristic measurement system (such as one or more of the systems described herein) consolidates comparisons at two or more locations of the user sensed by the corresponding first and second sensor assemblies 204 (or 104) and identifies the physiological characteristic such as the movement of a limb (gesture or the like) according to the comparison of each of the deformations to corresponding thresholds and interpretation of those comparisons by the controller 502 (e.g., the identification module 505). As described herein, in examples the physiological characteristic is identified as one or more of gestures of the limb (or digits) or portion of the user corresponding to the location of the sensor assemblies 204 on the body.

Several options for the method 600 follow. In one example, sensing the first deformation and the second deformation includes sensing the first deformation with at least a piezo element such as one or more first, second or third piezo elements 312, 314, 316 at the first location (e.g., one or more of the wrist, forearm, elbow, shoulder or the like) and sensing the second deformation includes sensing the deformation with at least another piezo element of a second sensor assembly 204 (or 104) at a second location for instance the second location corresponding to a different location on the body. In another example, sensing one or more of the first deformation or the second deformation includes sensing the deformation of a garment such as one or more of the garments 100, 200 shown herein coupled with the first and second sensor assemblies 204 (or 104). The garment 100, 200 is deformed by one or more of the first or second physiological movements. That is to say, with physiological movements such as motion of the limbs (or digits), beating of the heart, respiration by way of the lungs, the sensor elements including for instance one or more piezo elements 312, 314, 316 are deformed and accordingly the physiological movement is measured by these elements and interpreted by the controller, such as the controller 502.

In another example, the first location for the first sensor assembly is a first body location on a body such as one or more of a wrist 210, forearm 212, elbow 214, shoulder 216 or a torso location for instance the location corresponding to the chest or the area over one or more of the lungs or heart. In another example, the second location is a second body location on the body different from the first body location for instance one or more of other locations including for instance the shoulder 212, elbow 214, forearm 212, wrist 210 or another portion of the torso. Sensing of the first and second deformations according to the method 600 is conducted at each of the first and second body locations.

In still another example, one or more of the first or second sensory assemblies 204, 104 includes a first piezo element 312 at a first orientation and a second piezo element 314 at a second orientation different than the first orientation, for instance including but not limited to orientations such as along an x axis and a y axis, respectively. Sensing one or more of the first deformation of the first sensor assembly 204 or the second deformation of the second sensor assembly 204 (or 104) includes sensing a first component of the first or second deformation with the first piezo element 312 and sensing the second component of the same first or second deformation with a second piezo element 314. In another example, determining one or more of the type of the physiological characteristic and the characteristic magnitude of the physiological characteristic includes determining a deformation magnitude and a deformation direction (e.g., a vector) of one or more of the first or second deformations based on the first and second components.

In still another example, one or more of the first or second sensor assemblies 204 (or 104) includes a third piezo element 316 at a third orientation different than the first or second orientations and the first or second piezo elements 212, 314 respectively. Sensing one or more of the first deformation of the first sensor assembly 204 or the second deformation of the second sensor assembly 204 (or 104) includes sensing a third component of the first or second deformations with the third piezo element 318. The method 600 further includes filtering noise from one or more of the first or second components of the first or second deformations based on the sensed third component. That is to say, in one example the third component sensed with the third piezo element 316 is used to filter noise from measurements of one or more of the first or second deformations. For instance, detection of wrinkling of a fabric or deformable substrate or the like is measured as part of a third component (wrinkling is also incorporated with the first and second component measurements). The third component is used by the controller to accordingly filter out noise such as wrinkling of the fabric or deformable substrate that is common to each of the first, second and third components to provide a cleaner signal and accordingly a more accurate identification of movement at the sensor assembly.

In one example, the physiological characteristic corresponds to a gesture such as gestures of an arm, hand, digit or the like. The first location recited in the method 600 corresponds to a first limb location and the second location is a second limb location. The first and second limb locations include one or more of locations on a limb or portions of the anatomy coupled with the limb (such as the shoulder, hand, digits of the hand or the like). Determining one or more of the type of the physiological characteristic and the characteristic magnitude (e.g., inclusive of at least magnitude or vector) includes determining the type of the gesture and the magnitude of the gesture. In another example, the physiological characteristic is a physiological organ characteristic including but not limited to one or more of respiration rate, volume of inhalation and exhalation, variation of rate, heart rate, identification of the action of one or more of the heart chambers, volume of blood pumped by the heart or each of the chambers of the heart, identification of blood regurgitation or the like. In such an example, at least one of the first or second locations is a torso location, for instance the location of the sensor assembly 104 in FIG. 5 or FIG. 1 with the sensor assembly positioned over the chest cavity of the garment 100. In such an example, determining one or more of the type of the physiological characteristic and the characteristic magnitude includes determining the type of the physiological organ characteristic (e.g., heart rate, respiration rate or other characteristics as described previously herein) and the magnitude of the physiological organ characteristic including for instance heart rate, respiration rate, volume or the like.

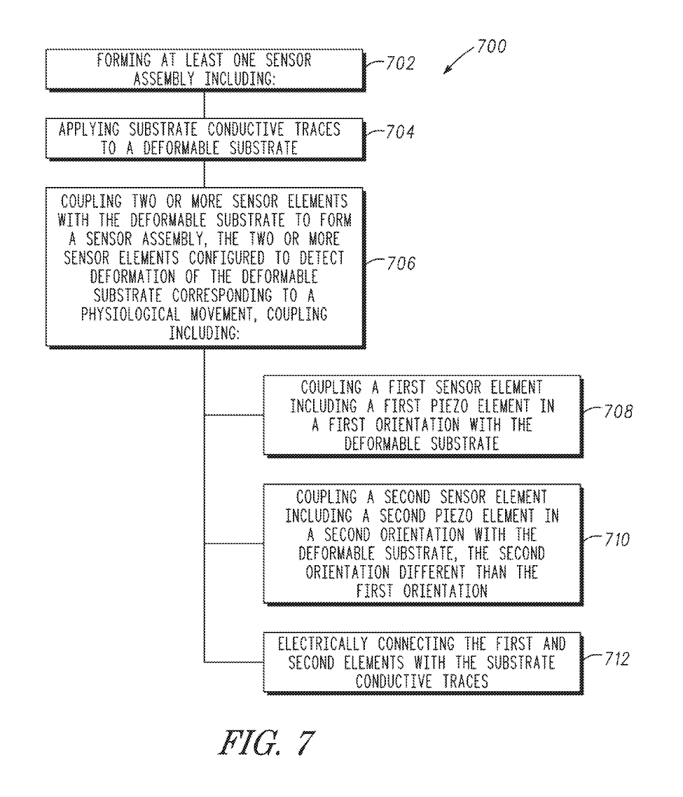

FIG. 7 shows one example of a method 700 for making a physiological character measurement system, such as the physiological characteristic measurement systems 102, 202, 502 previously described and shown herein. In describing the method 700 reference is made to one or more components, features, functions and steps previously described herein. Where convenient reference is made to the components, features, steps and the like with reference numerals. Reference numerals provided are exemplary and are nonexclusive. For instance components, features, functions, steps and the like described in the method 700 include but are not limited to the corresponding numbered elements provided herein, other corresponding features described herein (both numbered and unnumbered) as well as their equivalents.

At 702, the method 700 includes forming at least one sensor assembly, such as the sensor assembly 300 shown in FIG. 3. Forming the at least one sensor assembly 300 includes at 704 applying substrate conductive traces 320 to a deformable substrate 302. In one example, the deformable substrate includes but is not limited to an elastomeric substrate, a textile substrate, for instance one or more of the textiles of the garments 100, 200 (or the like).

At 706, the method 700 includes coupling one or more sensor elements (e.g., two or more and so on), for instance first and second sensor elements 306, 308, with the deformable substrate 302 to form a sensor assembly such as the sensor assembly 300. The one or more sensor elements 306, 308 are configured to detect deformation of the deformable substrate 302 (and corresponding deformation of elements, such as piezo element) corresponding to a physiological movement. Coupling of the two or more sensor elements includes, at 708, coupling a first sensor element including a first piezo element 312 at a first orientation (such as along an x axis) with the deformable substrate 302. At 710, the second sensor element 308 is coupled with the deformable substrate 302, and the second sensor element 308 includes a second piezo element 314 at a second orientation (such as along a y axis) with the deformable substrate 302. The second orientation is different than the first orientation

At 712, the method 700 includes electrically connecting the first and second sensor elements with substrate conductive traces such as the traces 320 previously shown and described in FIG. 3. In one example, the first and second sensor elements 306, 308 are coupled with the substrate conductive traces 320 to accordingly provide interconnection with interconnecting conductive traces 108, 208 provided on a garment 100, 200 as shown for instance in FIGS. 1 and 2.

Several options for the method 700 follow. In one example, applying the conductive traces such as one or more of the substrate conductive traces 320 and interconnecting conductive traces 108, 208 includes applying a conductive ink to a deformable substrate such as the deformable substrate 302 and curing the conductive ink. In another example, applying the conductive traces includes but is not limited to one or more of stenciling the conductive ink, screen printing the conductive ink, sputtering the conductive ink, patterning the conductive ink by lithography (e.g., by masking and etching), sewing conductive thread. In the example of a conductive ink the method 700 includes curing the conductive ink, for instance by way of allowing the conductive ink to set for a period of time or curing the conductive with heating.

In another example, coupling one or more of the first or second sensor elements includes applying a piezo ink to the deformable substrate 302 and then curing the piezo ink to accordingly form one or more of the piezo elements 312, 314, 316 (piezo resistive or piezoelectrical elements) on the deformable substrate 302. Coupling one or more of the first or second sensor elements 306, 308 (or the sensor element 310) consists of one or more of stenciling the piezo ink, screen printing the piezo ink, sputtering the piezo ink, patterning of the piezo ink by lithography (for instance masking and etching) and then curing the piezo ink for instance by way of ironing the piezo ink, allowing the piezo ink to set for a specified amount of time or heating the piezo ink in an oven or other heated environment to cure the piezo ink on the deformable substrate 302.

In yet another example, the method 700 includes encapsulating the substrate conductive traces 320 and the two or more sensor elements, for instance first, second and third sensor elements 306, 308, 310 shown in FIG. 3, within an encapsulant 408 (FIG. 4). In one example, the encapsulant includes an elastomer similar or identical to the elastomer used in the deformable substrate, such as the deformable substrate 402 shown in FIG. 4. The encapsulant is applied over the components of the sensor assembly to protect one or more of the conductive traces and sensor elements from weather, wear, washing or the like.

In yet another example, the deformable substrate includes an elastomer and the method 700 includes coupling at least one sensor assembly for instance one or more of the sensor assemblies 204 or 104 with at least a portion of a garment 100, 200 at a first location such as the first location corresponding to one or more of the anatomical locations on the garment 200 or a location for instance on the chest cavity for the garment 100. In another example, the system described herein includes at least one additional sensor assembly for instance one or more of the sensory assemblies 204, 104 and the method 700 includes coupling the other sensory assembly with at least another portion of the garment 100, 200 at a second location different than the first location. For instance the second location may correspond to a different portion of the anatomy for instance one or more of the wrist 210, forearm 212, elbow 214, shoulder 216 or the chest cavity as shown in one or more of FIGS. 1, 2 and 5.

In yet another example, the method 700 further includes coupling a controller, such as one or more of the controllers 102, 202, 502, with a garment such as the garments 100, 200. The controller is in communication with at least one of the sensor assemblies in the manner shown in FIG. 5. In another example, the method 700 includes interconnecting the at least one sensor assembly, such as one or more of the sensor assemblies 204, 104, with the controller with interconnecting conductive traces 108 shown in FIG. 1 (208 shown in FIG. 2) and FIG. 5. In one example, the interconnecting conductive traces 108, 208 are formed in a substantially similar manner to the conductive traces used in the sensory assemblies (e.g., by application of a conductive ink and followed by curing of the conductive ink). In another example, the interconnecting conductive traces include a conductive thread that is woven into the garment or formed as part of the garment.

### EXAMPLES

Example 1 can include subject matter such as can include a sensor assembly configured to monitor one or more physiological characteristics comprising: a deformable substrate, the deformable substrate includes a body side interface; substrate conductive traces coupled with the deformable substrate; and two or more physiological sensor elements coupled with the deformable substrate, the two or more physiological sensor elements include at least first and second sensor elements: the first sensor element includes a first piezo element in a first orientation along the deformable substrate, the first sensor element is electrically coupled with the substrate conductive traces, and the second sensor element includes a second piezo element in a second orientation along the deformable substrate different than the first orientation, the second sensor element is electrically coupled with the substrate conductive traces.

Example 2 can include, or can optionally be combined with the subject matter of Example 1, to optionally include an encapsulant, the two or more physiological sensor elements and the substrate conductive traces are surrounded by the encapsulant.

Example 3 can include, or can optionally be combined with the subject matter of one or any combination of Examples 1 or 2 to optionally include the deformable substrate includes an elastomer.

Example 4 can include, or can optionally be combined with the subject matter of one or any combination of Examples 1-3 to optionally include the deformable substrate includes a textile.

Example 5 can include, or can optionally be combined with the subject matter of one or any combination of Examples 1-4 to optionally include the deformable substrate consists of one or more of thermoplastic polyurethane, polydimethylsiloxane, silicone elastomers or butyl rubber.

Example 6 can include, or can optionally be combined with the subject matter of Examples 1-5 to optionally include at least one of the first and second piezo elements include a cured piezo-ink.

Example 7 can include, or can optionally be combined with the subject matter of Examples 1-6 to optionally include the substrate conductive traces include a cured conductive ink.

Example 8 can include, or can optionally be combined with the subject matter of Examples 1-7 to optionally include at least one of the first or second sensor elements includes a Wheatstone bridge, and the respective first or second piezo element is a component resistor of the Wheatstone bridge.

Example 9 can include, or can optionally be combined with the subject matter of Examples 1-8 to optionally include the body side interface is configured for coupling with a garment.

Example 10 can include, or can optionally be combined with the subject matter of Examples 1-9 to optionally include at least one sensor assembly as recited in example 1.

Example 11 can include, or can optionally be combined with the subject matter of Examples 1-10 to optionally include the garment consists of one or more of a clothing article, a cuff configured for positioning around a body part, a sleeve configured for positioning around a body part or a jewelry article.

Example 12 can include, or can optionally be combined with the subject matter of Examples 1-11 to optionally include a physiological characteristic measurement system comprising: a deformable substrate in the shape of at least a portion of a garment; two or more sensor assemblies coupled with the deformable substrate, the two or more sensor assemblies include at least first and second sensor assemblies: the first sensor assembly includes one or more first sensor elements including a piezo element coupled with the deformable substrate at a first location, and the piezo element is configured to detect a first deformation corresponding to a first physiological movement at the first location, and the second sensor assembly includes one or more second sensor elements including another piezo element coupled with the deformable substrate at a second location spaced from the first location, and the other piezo element is configured to detect a second deformation corresponding to a second physiological movement at the second location; and a controller in communication with each of the two or more sensor assemblies, and the controller includes: a comparison module configured to compare the detected first deformation with a first deformation threshold and compare the detected second deformation with a second deformation threshold, and an identification module configured to identify a physiological characteristic based on the compared first and second deformations.

Example 13 can include, or can optionally be combined with the subject matter of Examples 1-12 to optionally include the deformable substrate consists of one or more of a full garment, a shirt, a vest, a pant, a body suit, a coat, a sleeve or a cuff configured for reception on a limb.

Example 14 can include, or can optionally be combined with the subject matter of Examples 1-13 to optionally include the deformable substrate includes one or more elastomer substrates each with a body side interface configured for coupling with one or more of a garment or a user body.

Example 15 can include, or can optionally be combined with the subject matter of Examples 1-14 to optionally include interconnecting conductive traces interconnecting the two or more sensor assemblies with the controller.

Example 16 can include, or can optionally be combined with the subject matter of Examples 1-15 to optionally include the interconnecting conductive traces consist of one or more of cured conductive inks, conductive threads, conductive polymers, conductive bulk metal traces, or patterned traces.

Example 17 can include, or can optionally be combined with the subject matter of Examples 1-16 to optionally include the piezo element of at least the first sensor assembly includes: a first piezo element at a first orientation at the first location, and a second piezo element at a second orientation at the first location, and each of the first and second piezo elements are configured to detect the first deformation corresponding to the first physiological movement at the first location.

Example 18 can include, or can optionally be combined with the subject matter of Examples 1-17 to optionally include the first piezo element is configured to detect a first component of the first deformation parallel to the first orientation and the second piezo element is configured to detect a second component of the first deformation parallel to the second orientation.

Example 19 can include, or can optionally be combined with the subject matter of Examples 1-18 to optionally include the first orientation is orthogonal to the second orientation.

Example 20 can include, or can optionally be combined with the subject matter of Examples 1-19 to optionally include at least one of the piezo elements includes a cured piezo ink.

Example 21 can include, or can optionally be combined with the subject matter of Examples 1-20 to optionally include the physiological characteristic includes a user gesture having a type, direction and a magnitude, and the identification module is configured to identify one or more of the type of user gesture, the direction and the magnitude of the user gesture based on the compared first and second deformations.

Example 22 can include, or can optionally be combined with the subject matter of Examples 1-21 to optionally include a third sensor assembly having one or more third sensor elements including at least a third piezo element coupled with the deformable substrate at a third location, and the third piezo element is configured to detect a third deformation corresponding to a physiological organ characteristic at the third location, and the comparison module is configured compare the detected third deformation with a third deformation threshold, and the identification module is configured to identify the physiological organ characteristic based on the compared third deformation.

Example 23 can include, or can optionally be combined with the subject matter of Examples 1-22 to optionally include a method of making a physiological characteristic measurement system comprising: forming at least one sensor assembly including: applying substrate conductive traces to a deformable substrate including a body side interface; and coupling two or more sensor elements with the deformable substrate to form a sensor assembly, the two or more sensor elements configured to detect deformation of the deformable substrate corresponding to a physiological movement, coupling including: coupling a first sensor element including a first piezo element in a first orientation with the deformable substrate, coupling a second sensor element including a second piezo element in a second orientation with the deformable substrate, the second orientation different than the first orientation, and electrically connecting the first and second sensor elements with the substrate conductive traces.

Example 24 can include, or can optionally be combined with the subject matter of Examples 1-23 to optionally include applying the conductive traces includes: applying a conductive ink to the deformable substrate, and curing the conductive ink.

Example 25 can include, or can optionally be combined with the subject matter of Examples 1-24 to optionally include applying the conductive traces consists of one or more of curing a conductive ink, stenciling the conductive ink, screen printing the conductive ink, sputtering the conductive ink, ironing the conductive ink, patterning the conductive ink by lithography or sewing a conductive thread.

Example 26 can include, or can optionally be combined with the subject matter of Examples 1-25 to optionally include coupling one or more of the first sensor elements includes: applying a piezo-ink to the deformable substrate, curing the piezo-ink.

Example 27 can include, or can optionally be combined with the subject matter of Examples 1-26 to optionally include coupling one or more of the first sensor elements consists of one or more of curing a piezo-ink, stenciling the piezo-ink, screen printing the piezo-ink, sputtering the piezo-ink, patterning the piezo-ink by lithography or ironing the piezo-ink.

Example 28 can include, or can optionally be combined with the subject matter of Examples 1-27 to optionally include encapsulating the substrate conductive traces and the two or more sensor elements along the deformable substrate.

Example 29 can include, or can optionally be combined with the subject matter of Examples 1-28 to optionally include the deformable substrate includes an elastomer, and comprising coupling the at least one sensor assembly with at least a portion of a garment at a first location.

Example 30 can include, or can optionally be combined with the subject matter of Examples 1-29 to optionally include at least one sensor assembly includes another sensor assembly, and comprising coupling the other sensor assembly with at least another portion of the garment at a second location different than the first location.

Example 31 can include, or can optionally be combined with the subject matter of Examples 1-30 to optionally include coupling a controller with the garment, the controller in communication with the at least one sensor assembly.

Example 32 can include, or can optionally be combined with the subject matter of Examples 1-31 to optionally include interconnecting the at least one sensor assembly with the controller with interconnecting conductive traces extending along the garment.

Example 33 can include, or can optionally be combined with the subject matter of Examples 1-32 to optionally include a method for identifying a physiological characteristic comprising: sensing a first deformation of a first sensor assembly at a first location of a user, the first deformation corresponding to a first physiological movement; sensing a second deformation of a second sensor assembly at a second location of the user different than the first location, the second deformation corresponding to a second physiological movement; and identifying the physiological characteristic based on the sensed first and second deformations, identifying the physiological characteristic including: comparing the sensed first and second deformations to respective first and second deformation thresholds; and determining one or more of a type of the physiological characteristic and a characteristic magnitude of the physiological characteristic based on the comparisons of the first and second deformations to the respective first and second deformation thresholds.

Example 34 can include, or can optionally be combined with the subject matter of Examples 1-33 to optionally include sensing the first deformation and the second deformation includes: sensing the first deformation with at least a piezo element of the first sensor assembly at the first location, and sensing the second deformation with at least another piezo element of the second sensor assembly at the second location.

Example 35 can include, or can optionally be combined with the subject matter of Examples 1-34 to optionally include sensing one or more of the first deformation or the second deformation includes sensing the deformation of a garment coupled with the first and second sensor assemblies, the garment deformed by one or more of the first or second physiological movements.

Example 36 can include, or can optionally be combined with the subject matter of Examples 1-35 to optionally include the first location is a first body location on a body and the second location is a second body location on the body different from the first body location, and sensing the first deformation and sensing the second deformation includes: sensing the first deformation at the first body location, and sensing the second deformation at the second body location.

Example 37 can include, or can optionally be combined with the subject matter of Examples 1-36 to optionally include one or more of the first or second sensor assemblies include a first piezo element at a first orientation and a second piezo element at a second orientation different than the first orientation, and sensing one or more of the first deformation of the first sensor assembly or the second deformation of the second sensor assembly includes: sensing a first component of the first or second deformation with the first piezo element, and sensing a second component of the first or second deformation with the second piezo element.

Example 38 can include, or can optionally be combined with the subject matter of Examples 1-37 to optionally include determining one or more of the type of the physiological characteristic and the characteristic magnitude of the physiological characteristic includes determining a deformation magnitude and a deformation direction of one or more of the first or second deformations based on the first and second components.

Example 39 can include, or can optionally be combined with the subject matter of Examples 1-38 to optionally include one or more of the first or second sensor assemblies includes a third piezo element at a third orientation different than the first or second orientations, and sensing one or more of the first deformation of the first sensor assembly or the second deformation of the second sensor assembly includes: sensing a third component of the first or second deformation with the third piezo element, and filtering noise from one or more of the first or second components of the first or second deformation based on the sensed third component.

Example 40 can include, or can optionally be combined with the subject matter of Examples 1-39 to optionally include the physiological characteristic is a gesture, the first location is a first limb location and the second location is a second limb location, the first and second limb locations include one or more of locations on a limb or portions of the anatomy coupled with the limb, and determining one or more of the type of the physiological characteristic and the characteristic magnitude includes determining the type of the gesture and the magnitude of the gesture.

Example 41 can include, or can optionally be combined with the subject matter of Examples 1-40 to optionally include the physiological characteristic is a physiological organ characteristic, at least one of the first or second locations is a torso location, and determining one or more of the type of the physiological characteristic and the characteristic magnitude includes determining the type of the physiological organ characteristic and the magnitude of the physiological organ characteristic.

All features of the apparatuses described above (including optional features) may also be implemented with respect to the methods or processes described herein.

1. A sensor assembly configured to monitor one or more physiological characteristics comprising:
a deformable substrate, the deformable substrate includes a body side interface;
substrate conductive traces coupled with the deformable substrate; and
two or more physiological sensor elements coupled with the deformable substrate, the two or more physiological sensor elements include at least first and second sensor elements:
the first sensor element includes a first piezo element in a first orientation along the deformable substrate, the first sensor element is electrically coupled with the substrate conductive traces, and
the second sensor element includes a second piezo element in a second orientation along the deformable substrate different than the first orientation, the second sensor element is electrically coupled with the substrate conductive traces.

2. The sensor assembly of claim 1 comprising an ecapsulant, the two or more physiological sensor elements and the substrate conductive traces are surrounded by the encapsulant.

3. The sensor assembly of claim 1, the deformable substrate includes an elastomer.

4. The sensor assembly of claim 1, the deformable substrate includes a textile.

5. The sensor assembly of claim 1, the deformable substrate consists of one or more of thermoplastic polyurethane, polydimethylsiloxane, silicone elastomers or butyl rubber.

6. The sensor assembly of claim 1, at least one of the first and second piezo elements include a cured piezo-ink.

7. The sensor assembly of claim 1, the substrate conductive traces include a cured conductive ink.

8. The sensor assembly of claim 1, at least one of the first or second sensor elements includes a Wheatstone bridge, and the respective first or second piezo element is a component resistor of the Wheatstone bridge.

9. The sensor assembly of claim 1, the body side interface is configured for coupling with a garment.

10. A garment including at least one sensor assembly as recited in claim 1.

11. The garment of claim 10, the garment consists of one or more of a clothing article, a cuff configured for positioning around a body part, a sleeve configured for positioning around a body part or a jewelry article.

12. A physiological characteristic measurement system comprising:
a deformable substrate in the shape of at least a portion of a garment;
two or more sensor assemblies coupled with the deformable substrate, the two or more sensor assemblies include at least first and second sensor assemblies:
the first sensor assembly includes one or more first sensor elements including a piezo element coupled with the deformable substrate at a first location, and the piezo element is configured to detect a first deformation corresponding to a first physiological movement at the first location, and
the second sensor assembly includes one or more second sensor elements including another piezo element coupled with the deformable substrate at a second location spaced from the first location, and the other piezo element is configured to detect a second deformation corresponding to a second physiological movement at the second location; and
a controller in communication with each of the two or more sensor assemblies, and the controller includes:
a comparison module configured to compare the detected first deformation with a first deformation threshold and compare the detected second deformation with a second deformation threshold, and
an identification module configured to identify a physiological characteristic based on the compared first and second deformations.

13. The physiological characteristic measurement system of claim 12, the deformable substrate consists of one or more of a full garment, a shirt, a vest, a pant, a body suit, a coat, a sleeve or a cuff configured for reception on a limb.

14. The physiological characteristic measurement system of claim 12, the deformable substrate includes one or more elastomer substrates each with a body side interface configured for coupling with one or more of a garment or a user body.

15. The physiological characteristic measurement system of claim 12 comprising interconnecting conductive traces interconnecting the two or more sensor assemblies with the controller.

16. The physiological characteristic measurement system of claim 15, the interconnecting conductive traces consist of one or more of cured conductive inks, conductive threads, conductive polymers, conductive bulk metal traces, or patterned traces.

17. The physiological characteristic measurement system of claim 12, the piezo element of at least the first sensor assembly includes:
a first piezo element at a first orientation at the first location, and
a second piezo element at a second orientation at the first location, and each of the first and second piezo elements are configured to detect the first deformation corresponding to the first physiological movement at the first location.

18. The physiological characteristic measurement system of claim 17, the first piezo element is configured to detect a first component of the first deformation parallel to the first orientation and the second piezo element is configured to detect a second component of the first deformation parallel to the second orientation.

19. The physiological characteristic measurement system of claim 17, the first orientation is orthogonal to the second orientation.

20. The physiological characteristic measurement system of claim 17, at least one of the piezo elements includes a cured piezo ink.

21. The physiological characteristic measurement system of claim 12, the physiological characteristic includes a user gesture having a type, direction and a magnitude, and the identification module is configured to identify one or more of the type of user gesture, the direction and the magnitude of the user gesture based on the compared first and second deformations.

22. The physiological characteristic measurement system of claim 12 comprising a third sensor assembly having one or more third sensor elements including at least a third piezo element coupled with the deformable substrate at a third location, and the third piezo element is configured to detect a third deformation corresponding to a physiological organ characteristic at the third location, and
the comparison module is configured compare the detected third deformation with a third deformation threshold, and
the identification module is configured to identify the physiological organ characteristic based on the compared third deformation.

23. A method of making a physiological characteristic measurement system comprising:
forming at least one sensor assembly including:
applying substrate conductive traces to a deformable substrate including a body side interface; and
coupling two or more sensor elements with the deformable substrate to form a sensor assembly, the two or more sensor elements configured to detect deformation of the deformable substrate corresponding to a physiological movement, coupling including:
coupling a first sensor element including a first piezo element in a first orientation with the deformable substrate,
coupling a second sensor element including a second piezo element in a second orientation with the deformable substrate, the second orientation different than the first orientation, and
electrically connecting the first and second sensor elements with the substrate conductive traces.

24. The method of claim 23, applying the conductive traces includes:
applying a conductive ink to the deformable substrate, and curing the conductive ink.

25. The method of claim 23, applying the conductive traces consists of one or more of curing a conductive ink, stenciling the conductive ink, screen printing the conductive ink, sputtering the conductive ink, ironing the conductive ink, patterning the conductive ink by lithography or sewing a conductive thread.

26. The method of claim 23, coupling one or more of the first sensor elements includes:
applying a piezo-ink to the deformable substrate,
curing the piezo-ink.

27. The method of claim 23, coupling one or more of the first sensor elements consists of one or more of curing a piezo-ink, stenciling the piezo-ink, screen printing the piezo-ink, sputtering the piezo-ink, patterning the piezo-ink by lithography or ironing the piezo-ink.

28. The method of claim 23 comprising encapsulating the substrate conductive traces and the two or more sensor elements along the deformable substrate.

29. The method of claim 23, the deformable substrate includes an elastomer, and comprising coupling the at least one sensor assembly with at least a portion of a garment at a first location.

30. The method of claim 29, the at least one sensor assembly includes another sensor assembly, and comprising coupling the other sensor assembly with at least another portion of the garment at a second location different than the first location.

31. The method of claim 29 comprising coupling a controller with the garment, the controller in communication with the at least one sensor assembly.

32. The method of claim 31 comprising interconnecting the at least one sensor assembly with the controller with interconnecting conductive traces extending along the garment.
